(19) United States
(12) Patent Application Publication
(10) Pub. No.: US 2026/0117261 A1
(43) Pub. Date: Apr. 30, 2026

(54) METHOD FOR PREPARING RED MONASCUS PIGMENT AND ITS APPLICATIONS

(71) Applicant: HENAN ZHONGDA HENGYUAN BIOTECHNOLOGY STOCK CO., LTD., Luohe City (CN)

(72) Inventors: Lizhi CHEN, Luohe City (CN); Tianyi PAN, Luohe City (CN); Ziheng JIN, Luohe City (CN); Linzheng LI, Luohe City (CN); Yanjun WEN, Luohe City (CN)

(21) Appl. No.: 19/263,741

(22) Filed: Jul. 9, 2025

(30) Foreign Application Priority Data
Oct. 30, 2024 (CN) 202411534197.7

(51) Int. Cl. C12P 1/02; C09B 61/00

(52) U.S. Cl. CPC C12P 1/02; C09B 61/00

(57) ABSTRACT

The present invention pertains to the field of natural fermentation pigments and specifically relates to a method for preparing red Monascus pigment and its applications. The method disclosed herein enables an extraction yield of red Monascus pigment of more than 85%. Compared to red Monascus pigments obtained by conventional extraction techniques, the red Monascus pigment prepared by the present method exhibits significantly improved extraction yield, stability, and antioxidant capacity. In particular, the pigment possesses a deep red hue, making it especially suitable for use in baked goods. The improved properties of the pigment facilitate its broader applicability and commercial potential.

This application claims priority to Chinese Patent Application No. 202411534197.7, filed on Oct. 30, 2024, which is incorporated by reference for all purposes as if fully set forth herein.

### TECHNICAL FIELD

The present invention pertains to the field of natural fermentation pigments, and more specifically, to a method for preparing red Monascus pigment and its applications.

### BACKGROUND

Numerous studies have reported that almost all synthetic pigments do not provide any nutritional value to the human body. Research by the UK Food Standards Agency has shown that six artificial colorants, including the well-known Lemon Yellow and Sunset Yellow, can affect children's cognitive function. In severe cases, these colorants may lead to a decrease in children's IQ by up to 5.5 points. As people's understanding of the harmful effects of synthetic pigments deepens, natural pigments have increasingly gained attention. In contrast to synthetic pigments, edible natural pigments are not only non-toxic but some also possess nutritional value and even certain pharmacological effects. Currently, the development and use of natural pigments to replace synthetic colorants have become a growing trend in the food and cosmetics industries.

In recent years, an increasing number of reports have highlighted the toxicity, carcinogenic, and mutagenic effects of synthetic chemical colorants, causing public concern. Currently, both the production and export of synthetic colorants are declining. With growing market demand and regulatory restrictions, the use of synthetic colorants will continue to be increasingly restricted, especially in developed countries that are tightening regulations on their use. Red Monascus pigment is an ancient natural red colorant from China, known for its high safety, strong thermal stability, excellent affinity for proteins, vibrant red hue, stable quality, and low cost—qualities that surpass synthetic colorants and other natural pigments. It also exhibits potent physiological activities such as cholesterol-lowering and blood pressure-reducing effects, and has widespread applications in the food industry.

Monascus purpureus is an important dual-purpose microorganism in China, and its fermented product, red Monascus, has been used in China for over a thousand years, with applications in food coloring, pharmaceuticals, brewing, and other industries. Monascus purpureus is capable of producing a variety of beneficial secondary metabolites, including red Monascus pigment, Monacolin K, γ-aminobutyric acid, ergosterol, and sterols. Since red Monascus pigment is a mixture containing various coloring substances, it tends to exhibit an orange-red hue. Additionally, due to the presence of conjugated double bonds in its components, it is prone to decomposition and fading, which has always been one of the factors limiting its stability and hindering the development of the industry. Therefore, the search for cost-effective color stabilizers that offer good color protection while meeting national food safety standards, to reduce costs and produce high-quality red Monascus pigment, is a current challenge for enterprises. Meanwhile, in practical product applications, there is an increasing demand for a wider range of color shades, and the limited stability and single color tone of the pigment restrict its practical use and development in the food industry to some extent.

Numerous Chinese patents have proposed methods to enhance the stability of red Monascus pigment. Chinese Patent CN 102702785 A discloses a method for preparing red Monascus pigment with superior light stability by using condensed tannins in the fermentation medium, which inhibits color fading caused by light exposure, thereby producing a light-stable composite red Monascus pigment. Chinese Patent CN 103882063 A discloses a preparation method for a red Monascus pigment with high light stability, wherein Monascus purpureus is used as the strain for liquid deep fermentation. After fermentation, the resulting cake is extracted with an organic solvent, with the addition of Vitamin C and lychee seed extract during the extraction process. The red Monascus pigment solution is filtered and dried to obtain the red Monascus pigment powder, which exhibits high light stability and maintains its color even under prolonged exposure to sunlight and artificial light. Chinese Patent CN 101530190 B discloses a color stabilizer for food-grade red Monascus pigment, which consists of 5%-10% catechins, 10%-25% Vitamin C, 30%-40% glucose, with the remainder being rutin. This stabilizer, as a food additive, can be applied to the surface of food to make the red Monascus pigment resistant to high temperatures and oxidation, while maintaining its color even under prolonged exposure to sunlight and artificial light. Patent CN 104195178 A provides a method for preparing high-color-tone red Monascus pigment by adding monosodium glutamate to the extracted intracellular red Monascus pigment in a surfactant micelle solution, or by adding surfactants and monosodium glutamate to the fermentation medium, thus converting the red Monascus orange pigment into high-tone, high-color-value red Monascus pigment with a low orange pigment content.

Most of the above patents focus on the addition of color stabilizers, which improve the stability of red Monascus pigment to some extent but do not enhance the extraction yield or address the issue of color tone. Currently, no patent has been found that addresses both the red Monascus pigment's color tone and extraction yield. Additionally, there is no method that can simultaneously increase the yield while achieving a deeper red hue and higher stability in the product.

### SUMMARY OF THE INVENTION

In view of this, the technical problem to be solved by the present invention is to provide a method for preparing red Monascus pigment and its applications. The method provided by the present invention is simple to operate, has a high extraction yield, produces a stable product, and results in a red Monascus pigment with a deeper red hue.

The present invention provides a method for preparing red Monascus pigment, comprising the following steps:
Step 1: Obtain a Monascus purpureus strain, cultivate, ferment, acid precipitate, and perform the first filtration to obtain a precipitate;
Step 2: Wash the precipitate obtained in Step 1, perform a second filtration to obtain a filter cake;
Step 3: Extract the filter cake obtained in Step 2 with an ethanol solution, perform peptide powder extraction, and then perform a third filtration to obtain the filtrate;
Step 4: Perform a fourth filtration, concentrate, and dry the filtrate obtained in Step 3 to obtain red Monascus pigment powder.

In some Examples, in Step 1, the cultivation temperature is between 30° C. and 35° C., and the cultivation time is between 6 and 7 days.

In some specific embodiments, in Step 1, the cultivation temperature is 30° C., and the cultivation time is 6.5 days.

In some Examples, in Step 1, the fermentation medium comprises: 8 wt % to 10 wt % enzyme-hydrolyzed rice flour, 1 wt % to 3 wt % soybean powder, 0.3 wt % to 1 wt % corn slurry, 0.1 wt % to 0.2 wt % sodium nitrate, 0.1 wt % to 0.3 wt % KH₂PO₄, and 0.05 wt % to 0.3 wt % MgSO₄, wherein the fermentation temperature is between 30° C. and 35° C., and the fermentation time is between 5 and 8 days.

In some specific embodiments, the fermentation medium comprises: 9.5 wt % enzyme-hydrolyzed rice flour, 2.1 wt % soybean powder, 0.6 wt % corn slurry, 0.18 wt % sodium nitrate, 0.2 wt % KH₂PO₄, and 0.1 wt % MgSO₄, wherein the fermentation temperature is 32° C., and the fermentation time is 7 days.

In some Examples, the method for preparing enzyme-hydrolyzed rice flour comprises mixing rice flour and amylase at a mass ratio of 1:(0.005 to 0.01), performing enzymolysis, cooling, and then adjusting the pH to 3.8 to 4.0 using lactic acid, wherein:

the amylase is medium-temperature amylase with an enzyme activity of 2000 U/mL, the enzymolysis temperature is between 50° C. and 60° C., and the enzymolysis time is between 30 and 60 minutes.

In some specific embodiments, the mass ratio of rice flour to amylase is 1:0.01, the enzymolysis temperature is 60° C., and the enzymolysis time is between 30 and 40 minutes.

In some Examples, the acid precipitation includes adjusting the pH of the fermentation broth to between 3.0 and 3.5 using hydrochloric acid, and the acid precipitation time is between 2 and 5 hours.

In some specific embodiments, the acid precipitation includes adjusting the pH of the fermentation broth to 3.0, 3.2, or 3.5 using hydrochloric acid, and the acid precipitation time is 3 hours.

In some Examples, in Step 2, the washing process includes mixing the precipitate with an ethanol solution having a volume fraction of 20% to 40% and stirring for 2 to 3 hours.

In some specific embodiments, the washing process includes mixing the precipitate with an ethanol solution having a volume fraction of 25%, 35%, or 40% and stirring for 3 hours.

In some Examples, in Step 3, during the ethanol solution extraction, the volume fraction of ethanol in the solution is between 70% and 85%, the mass-to-volume ratio of the filter cake to the ethanol solution is 1:(8 to 10), and the extraction time is between 2 and 3 hours.

In some specific embodiments, the volume fraction of ethanol in the solution is 80%, the mass-to-volume ratio of the filter cake to the ethanol solution is 1:8, and the extraction time is 3 hours.

In some specific embodiments, during the ethanol solution extraction, the volume fraction of ethanol in the solution is 75%, the mass-to-volume ratio of the filter cake to the ethanol solution is 1:10, and the extraction time is 2 hours.

In some specific embodiments, during the ethanol solution extraction, the volume fraction of ethanol in the solution is 80%, the mass-to-volume ratio of the filter cake to the ethanol solution is 1:10, and the extraction time is 2 hours.

In some Examples, in Step 3, the peptide powder extraction includes adding peptide powder to the ethanol solution either during or after the ethanol solution extraction, followed by mixing, adjusting the pH to 7 to 9 with sodium hydroxide, and stirring for 5 to 8 hours. The peptide powder includes at least one of corn oligopeptides, wheat oligopeptides, bovine bone peptides, collagen peptides, hydrolyzed fish peptides, and soybean peptides, and the amount of peptide powder added is 20% to 50% of the weight of the filter cake.

In some specific embodiments, the peptide powder is soybean peptide, the pH of the mixed solution is adjusted to 8.0 with sodium hydroxide, and stirring extraction is performed for 6 hours, with the amount of peptide powder added being 20% of the weight of the filter cake.

In some specific embodiments, the peptide powder is corn oligopeptide, the pH of the mixed solution is adjusted to 7.5 with sodium hydroxide, and stirring extraction is performed for 5 hours, with the amount of peptide powder added being 25% of the weight of the filter cake.

In some specific embodiments, the peptide powder is freshwater fish collagen peptide, the pH of the mixed solution is adjusted to 8.0 with sodium hydroxide, and stirring extraction is performed for 5 hours, with the amount of peptide powder added being 25% of the weight of the filter cake.

In some Examples, in Step 4, the fourth filtration is performed using diatomaceous earth filtration, and the concentration process includes reverse osmosis concentration and/or vacuum concentration.

In some specific embodiments, the reverse osmosis concentration includes membrane concentration, and the concentration process involves sequentially applying membrane concentration and vacuum concentration to the filtrate obtained after the fourth filtration.

The present invention provides red Monascus pigment obtained by the preparation method described herein. The extraction yield of the red Monascus pigment is ≥85%; the Lab color values satisfy: L*≥67, a*≥46, b*≥25; after light exposure, the pigment retention rate is ≥46%; and after high-temperature treatment (120° C., 40 minutes), the retention rate is ≥52%.

The present invention provides the application of the red Monascus pigment obtained by the method described herein as an additive in the preparation of food and/or cosmetics.

The present invention provides a method for preparing red Monascus pigment and the red Monascus pigment obtained by the method. The extraction yield of the red Monascus pigment produced by the method of the present invention exceeds 85%, and compared to red Monascus pigment extracted using traditional methods, the pigment obtained by the method of the present invention shows significantly improved yield, stability, and antioxidant properties. In particular, the pigment has a deeper red hue, making it more suitable for use in pastries, which is beneficial for the widespread application and promotion of red Monascus pigment.

### DESCRIPTION OF THE DRAWING

FIG. 1 shows a comparison of the appearance of the extracted filtrates recovered from Example 2 (left) and Example 4 (right).

### DETAILED DESCRIPTION OF EMBODIMENTS

The present invention provides a method for preparing red Monascus pigment and its applications. Those skilled in the art can refer to the content of this document and appropriately modify process parameters for implementation. It is particularly noted that all similar substitutions and modifications are apparent to those skilled in the art and are considered to be included within the scope of the present invention. The method and applications of the present invention have been described through preferred embodiments, and relevant individuals can clearly make changes or appropriate modifications and combinations to the methods and applications described herein without departing from the content, spirit, and scope of the present invention to realize and apply the invention.

The materials used in the present invention are all commercially available products and can be purchased on the market. The specific details are as follows:
The Monascus purpureus strain is of the species Monascus purpureus;
The medium-temperature amylase is sourced from Taian Xindeli Biotechnology Co., Ltd.;
Corn oligopeptide is sourced from Zhongshi Duqing (Shandong) Biotechnology Co., Ltd.;
Wheat oligopeptide is sourced from Zhongshi Duqing (Shandong) Biotechnology Co., Ltd.;
Bovine bone peptide is sourced from Dezhou Lanli Biotechnology Co., Ltd.;
Collagen peptide 1 is sourced from Qingdao Wantuming Biological Products Co., Ltd.;
Hydrolyzed fish peptide is sourced from Hainan Yuanpeptide Biotechnology Co., Ltd.;
Soybean peptide powder 1 is sourced from Dezhou Lanli Biotechnology Co., Ltd.;
Soybean peptide powder 2 is sourced from Zhongshi Duqing (Shandong) Biotechnology Co., Ltd.;
Wheat oligopeptide 2 is sourced from Zhongshi Duqing (Shandong) Biotechnology Co., Ltd.;
Soybean peptide powder 3 is sourced from Shandong Tianjiu Biotechnology Co., Ltd.;
Collagen peptide 2 is sourced from Jiaxing Mingsheng Biotechnology Co., Ltd.;
Amino acids are sourced from Hebei Changhao Biotechnology Co., Ltd.

The following examples further illustrate the present invention.

### Example 1

Method for preparing deep red hue and highly stable red Monascus pigment of the present invention:

(1) Cultivation: Inoculate the Monascus purpureus strain onto a sterilized slant culture medium and incubate in an incubator at 30° C. for 6.5 days.

(2) Fermentation: Inoculate the Monascus purpureus strain obtained in step (1) into the fermentation medium, and shake incubate at 32° C. for 7 days. The fermentation formula consists of: 9.5% rice flour, 2.1% soybean powder, 0.6% corn slurry, 0.18% sodium nitrate, 0.2% KH₂PO₄, and 0.1% MgSO₄. Rice flour enzymolysis: After mixing the ingredients, add 0.5% medium-temperature amylase (enzyme activity 2000 U/mL), stir and enzymolyze at 60° C. for 30 minutes. Add the other ingredients, cool, and adjust the pH to 4.0 using lactic acid.

(3) Filtration: Add hydrochloric acid to the fermentation broth from step (2), stir, and adjust the pH to 3.0. Precipitate for 3 hours and filter to obtain the filter cake.

(4) Washing: After grinding the filter cake obtained in step (3), dissolve it in 25% alcohol solution, stir for 3 hours, and perform a second filtration. Collect the washed filter cake.

(5) Extraction: Add 8 times the amount of 80% ethanol to the filter cake from step (4) and stir for 3 hours. Add a water solution of food-grade soybean peptide powder 1, with the amount of peptide powder added being 20% of the weight of the filter cake. Adjust the pH of the ethanol solution to 8.0 using NaOH, stir for 6 hours for extraction, and then filter to recover the filtrate.

(6) Membrane concentration: After diatomaceous earth filtration, the filtrate from step (5) enters a membrane concentration device.

(7) Recovery: The extract from step (6) is subjected to secondary concentration using a vacuum rotary evaporator to recover the alcohol-free purified liquid.

(8) Drying: Spray-dry the purified liquid to obtain red Monascus pigment powder.

### Example 2

Method for preparing deep red hue and highly stable red Monascus pigment of the present invention:

(1) Cultivation: Inoculate the Monascus purpureus strain onto a sterilized slant culture medium and incubate in an incubator at 30° C. for 6.5 days.

(2) Fermentation: Inoculate the Monascus purpureus strain obtained in step (1) into the fermentation medium, and shake incubate at 32° C. for 6.5 days. The fermentation formula consists of: 9.5% rice flour, 2.1% soybean powder, 0.6% corn slurry, 0.18% sodium nitrate, 0.2% KH₂PO₄, and 0.1% MgSO₄. Rice flour enzymolysis: After mixing the ingredients, add 0.6% medium-temperature amylase (enzyme activity 2000 U/mL), stir and enzymolyze at 60° C. for 40 minutes. Add the other ingredients, cool, and adjust the pH to 3.85 using lactic acid.

(3) Filtration: Add hydrochloric acid to the fermentation broth from step (2), stir, and adjust the pH to 3.5. Precipitate for 3 hours and filter to obtain the filter cake.

(4) Washing: After grinding the filter cake obtained in step (3), dissolve it in a 35% alcohol solution, stir for 3 hours, and perform a second filtration. Collect the washed filter cake.

(5) Extraction: Add 10 times the amount of 75% ethanol to the filter cake from step (4) and stir for 2 hours. Add a food-grade corn oligopeptide powder solution, with the amount of peptide powder added being 25% of the weight of the filter cake. Adjust the pH of the ethanol solution to 7.5 using NaOH, stir for 5 hours for extraction, and then filter to recover the filtrate.

(6) Membrane concentration: After diatomaceous earth filtration, the filtrate from step (5) enters a membrane concentration device.

(7) Recovery: The extract from step (6) is subjected to secondary concentration using a vacuum rotary evaporator to recover the alcohol-free purified liquid.

(8) Drying: Spray-dry the purified liquid to obtain red Monascus pigment powder.

### Example 3

Method for preparing deep red hue and highly stable red Monascus pigment of the present invention:

(1) Cultivation: Inoculate the Monascus purpureus strain onto a sterilized slant culture medium and incubate in an incubator at 30° C. for 6.5 days.

(2) Fermentation: Inoculate the Monascus purpureus strain obtained in step (1) into the fermentation medium, and shake incubate at 32° C. for 7 days. The fermentation formula consists of: 9.5% rice flour, 2.1% soybean powder, 0.6% corn slurry, 0.18% sodium nitrate, 0.2% KH₂PO₄, and 0.1% MgSO₄. Rice flour enzymolysis: After mixing the ingredients, add 0.5% medium-temperature amylase (enzyme activity 2000 U/mL), stir and enzymolyze at 60° C. for 30 minutes. Add the other ingredients, cool, and adjust the pH to 3.95 using lactic acid.

(3) Filtration: Add hydrochloric acid to the fermentation broth from step (2), stir, and adjust the pH to 3.2. Precipitate for 3 hours and filter to obtain the filter cake.

(4) Washing: After grinding the filter cake obtained in step (3), dissolve it in a 40% alcohol solution, stir for 3 hours, and perform a second filtration. Collect the washed filter cake.

(5) Extraction: Add 10 times the amount of 80% ethanol to the filter cake from step (4) and stir for 2 hours. Add a food-grade fish collagen peptide powder solution, with the amount of peptide powder added being 25% of the weight of the filter cake. Adjust the pH of the ethanol solution to 8.0 using NaOH, stir for 5 hours for extraction, and then filter to recover the filtrate.

(6) Membrane concentration: After diatomaceous earth filtration, the filtrate from step (5) enters a membrane concentration device.

(7) Recovery: The extract from step (6) is subjected to secondary concentration using a vacuum rotary evaporator to recover the alcohol-free purified liquid.

(8) Drying: Spray-dry the purified liquid to obtain red Monascus pigment powder.

### Example 4

A method for preparing conventional red Monascus pigment:

(1) Cultivation: Inoculate the Monascus purpureus strain onto a sterilized slant culture medium and incubate in an incubator at 30° C. for 6.5 days.

(2) Fermentation: Inoculate the Monascus purpureus strain obtained in step (1) into the fermentation medium, and shake incubate at 33° C. for 6 to 7 days. The fermentation formula consists of: 9.5% rice flour, 2.1% soybean powder, 0.6% corn slurry, 0.18% sodium nitrate, 0.2% KH₂PO₄, and 0.1% MgSO₄. Rice flour enzymolysis: After mixing the ingredients, add 0.6% medium-temperature amylase (enzyme activity 2000 U/mL), stir and enzymolyze at 60° C. for 30 minutes. Add the remaining ingredients, cool, and adjust the pH to 3.85 using lactic acid.

(3) Filtration: Add hydrochloric acid to the fermentation broth from step (2), stir, and adjust the pH to 3.0. Precipitate for 3.5 hours and filter to obtain the filter cake.

(4) Extraction: Add 8 times the amount of 80% ethanol to the filter cake from step (3), stir for 2 hours, adjust the pH of the ethanol solution to 7.5 using NaOH, stir for 3 hours for extraction, and then filter to recover the filtrate.

(5) Membrane concentration: After diatomaceous earth filtration, the filtrate from step (4) enters a membrane concentration device.

(6) Recovery: The extract from step (5) is subjected to secondary concentration using a vacuum rotary evaporator to recover the alcohol-free purified liquid.

(7) Drying: Spray-dry the purified liquid to obtain red Monascus pigment powder.

The color value of red Monascus pigment is determined according to the method specified in GB 1886.181-2016.

Color tone evaluation: Prepare a water solution of red Monascus pigment at a certain concentration, adjust the solution to an absorbance of A=1.00, and measure the color tone using a colorimeter.

Light stability test: Place the solution at a certain concentration in a light exposure chamber and measure the retention rate of the red Monascus pigment after 48 hours of exposure.

Thermal stability test: Under the same absorbance conditions, heat the solution to 120° C. for 40 minutes and measure the thermal stability of the red Monascus pigment.

The specific experimental data are shown in Table 1 below.

TABLE 1

| | hue | | | Extraction | Light | Thermal |
|---|---|---|---|---|---|---|
| | L* | a* | b* | yield | stability | stability |
| EXAMPLE 1 | 68.82 | 46.61 | 26.67 | 85.51% | 50.66% | 54.18% |
| EXAMPLE 2 | 68.11 | 46.77 | 25.74 | 86.40% | 46.51% | 52.63% |
| EXAMPLE 3 | 67.88 | 46.78 | 25.57 | 85.20% | 47.34% | 54.25% |
| EXAMPLE 4 | 71.42 | 43.09 | 34.21 | 70.50% | 33.21% | 46.50% |

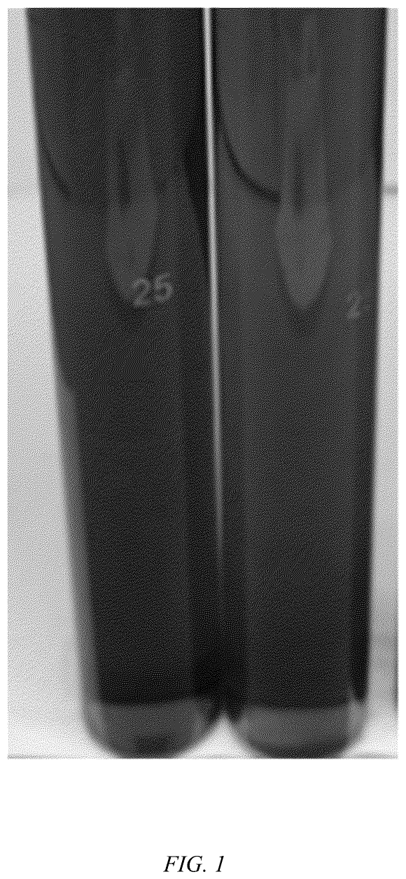

(1) The experimental data show that the addition of peptide powder during the extraction process results in a deeper red hue, with a higher a (redness) value and a* lower b* (yellowness) value. The color tone is deep red, which can meet the needs of different industries. Additionally, since the hue is redder, the amount of pigment required can be reduced.
(2) Compared to Examples 1-3, Example 4 shows a significantly higher extraction yield, which saves costs and reduces resource waste.
The appearance comparison of the extracted filtrates recovered from Example 2 and Example 4 is shown in FIG. 1.

Compared to Example 4, Examples 1-3, which include the addition of peptide powder, show a significant improvement in the stability of the red Monascus pigment, which is clearly superior to conventional red Monascus pigment, benefiting its use for food coloring and stability

### Example 5

To further verify the optimal amount of peptide used in the extraction of red Monascus pigment, in this embodiment, the extraction step is the same as in Example 1. In the extraction step, collagen peptide GDB-30sp is added at different ratios. The optimal amount of peptide for the extraction process is determined through repeated verification, and color difference analysis is performed (E1%=5 for a 0.5% aqueous solution, concentrated solution). The results are shown in Table 2 below.

TABLE 2

| | | | | Maximum | |
|---|---|---|---|---|---|
| Amount | | | | absorption | |
| added | L* | a* | b* | (nm) | Yield |
| 5% | 42.54 | 60.58 | 51.02 | 501 | 70.56% |
| 10% | 43.98 | 63.12 | 52.12 | 505 | 75.85% |
| 15% | 44.65 | 64.87 | 52.52 | 511 | 82.5% |
| 20% | 46.52 | 66.04 | 54.56 | 515 | 85.41% |
| 25% | 47.04 | 66.51 | 55.19 | 515 | 84.98% |
| 30% | 48.56 | 66.38 | 55.65 | 515 | 85.55% |
| 35% | 48.66 | 66.49 | 55.75 | 516 | 85.2% |
| 40% | 48.68 | 66.85 | 56.04 | 516 | 85.8% |
| 50% | 48.59 | 66.68 | 55.81 | 516 | 85.1% |

The results show that as the amount of collagen peptide increases, the hue becomes significantly redder, with no significant increase after reaching 20%. The color tone brightens, and the extraction effect is poor below 20%, with a noticeably grayish tone. This may be because the peptide amount is insufficient to bind with the pigment to produce color. As the peptide amount increases, the extraction yield improves significantly, remaining consistent between 20% and 50%, with no further increase. When the peptide amount exceeds 20%, the color tone continues to brighten, and the redness value (a*) no longer increases, indicating that the orange pigments capable of binding and producing color have been fully converted to red. Considering the price and cost of the peptide, the optimal amount of addition is between 20% and 50%, and further increases in the addition are not meaningful.

### Example 6

To further verify the effect of different peptide powders and manufacturers on the extraction efficiency, in this example, the extraction step is the same as in Example 1. In the extraction step, different types of protein peptides listed in Table 5 were used for extraction. The results are shown in Table 5.

TABLE 5

| | | | Color difference (E1% = 5 | | | |
|---|---|---|---|---|---|---|
| Varieties of protein | | Total | for a 0.5% aqueous solution) | | | Extraction |
| peptides | λMax | E495 | L* | a* | b* | yield |
| Corn oligopeptides | 507 nm | 12300 | 51.01 | 66.88 | 56.36 | 87.85% |
| Wheat oligopeptides | 509 nm | 11540 | 49.23 | 66.63 | 55.52 | 82.43% |
| Bovine bone peptides | 514 nm | 11330 | 47.54 | 66.78 | 53.75 | 80.93% |
| Collagen peptide 1 | 515 nm | 11380 | 47.22 | 66.83 | 53.04 | 81.29% |
| Hydrolyzed fish peptides | 511 nm | 11360 | 48.54 | 66.52 | 55.79 | 81.14% |
| Soybean peptide powder 1 | 513 nm | 11430 | 46.46 | 66.80 | 53.57 | 81.64% |
| Soybean peptide powder 2 | 514 nm | 11720 | 47.35 | 66.86 | 53.69 | 83.71% |
| Wheat oligopeptide 2 | 510 nm | 11570 | 48.97 | 66.78 | 54.79 | 82.64% |
| Soybean peptide powder 3 | 509 nm | 12020 | 47.21 | 66.40 | 54.03 | 85.86% |
| Collagen peptide 2 | 511 nm | 12250 | 47.51 | 66.71 | 56.16 | 87.50% |

The results show that different types of peptides exhibit variations in color tone, though the differences are not significant, with all showing a deep red hue. However, there are differences in extraction yield, with relatively larger variations. Collagen peptide 2 and corn oligopeptide showed better results.

### Example 7

To investigate the effect of different ethanol concentrations on the extraction of filter cake, in this embodiment, the extraction step is the same as in Example 1. In the extraction step, ethanol solutions with different concentrations, as shown in Table 6, were used for extraction. The results are shown in Table 6.

TABLE 6

| Ethanol | Vacuum | | Extraction | Color difference | | | |
|---|---|---|---|---|---|---|---|
| concentration | filtration | Recovery | yield | L* | a* | b* | Note |
| 20% | Difficulty | Difficult | 66.27% | 67.45 | 47.83 | 24.75 | Cloudy |
| | in vacuum | to | | | | | solution |
| | filtration, | process, | | | | | |
| | centrifugation | relatively | | | | | |
| 35% | Difficulty | slow | 65.50% | 68.25 | 47.36 | 25.06 | |
| | in vacuum | | | | | | |
| | filtration, | | | | | | |
| | centrifugation | | | | | | |
| 50% | Vacuum | | 63.30% | 69.19 | 46.95 | 26.64 | Clear |
| | filtration is | | | | | | solution |
| | slow | | | | | | |
| 60% | Vacuum | Normal | 76.63% | 68.79 | 47.19 | 26.74 | |
| | filtration is | | | | | | |
| | slow | | | | | | |
| 70% | Vacuum | | 85.62% | 68.79 | 46.95 | 26.82 | |
| 80% | filtration is | | 85.39% | 68.59 | 47.02 | 26.75 | |
| 85% | fast and | | 85.06% | 68.66 | 47.06 | 26.88 | |
| | efficient | | | | | | |

The results show that with a low alcohol concentration, the pigment extraction is incomplete, filtration is difficult, and there are many impurities in the extract, resulting in a turbid solution. With higher alcohol concentrations, extraction above 70% operates normally, and the extraction yield exceeds 85%. Further increases in alcohol concentration do not significantly improve the extraction yield.

### Example 8

To further investigate the effect of different amino acid additions on pigment extraction, in this embodiment, the extraction step is the same as in Example 1. In the extraction step, different types of amino acids listed in Table 7 were used for extraction. The total amount of amino acids added was kept constant, and the amount added was calculated based on the molecular weight of each amino acid. The results are shown in Table 7.

TABLE 7

| | Amount | Color difference | | | Extraction | |
|---|---|---|---|---|---|---|
| Amino acid types | added | L* | a* | b* | yield | Note |
| Blank | / | 71.08 | 43.01 | 33.58 | 71.05% | / |
| Glycine | 0.075% | 69.58 | 44.85 | 28.95 | 75.50% | / |
| Taurine | 0.125% | 68.65 | 44.25 | 29.05 | 78.65% | / |
| Arginine | 0.174% | 69.98 | 43.78 | 30.58 | 76.09% | / |
| Histidine | 0.155% | 72.55 | 40.25 | 36.88 | 78.05% | Yellowish |
| Methionine | 0.149% | 73.85 | 41.56 | 38.65 | 72.05% | has an -s odor |
| Aspartic acid | 0.133% | 71.24 | 43.51 | 33.41 | 77.01% | poorly soluble, soluble in water |
| Tyrosine | 0.181% | 71.52 | 44.02 | 31.02 | 80.14% | / |
| Cysteine | 0.121% | 72.05 | 44.24 | 32.01 | 81.05% | / |
| Lysine | 0.146% | 71.85 | 44.03 | 33.09 | 79.85% | / |
| Serine | 0.105% | 72.09 | 44.81 | 32.01 | 81.56% | / |
| Threonine | 0.119% | 72.51 | 43.41 | 34.51 | 81.56% | / |
| Valine | 0.117% | 73.01 | 43.54 | 35.04 | 80.64% | / |
| Leucine | 0.131% | 72.86 | 43.85 | 34.89 | 83.05% | / |
| Isoleucine | 0.131% | 72.66 | 43.85 | 35.01 | 81.09% | / |
| Monosodium glutamate | 0.147% | 73.05 | 43.65 | 35.33 | 82.69% | / |
| Proline | 0.115% | 73.61 | 42.98 | 36.65 | 76.54% | / |
| Phenylalanine | 0.165% | 72.74 | 44.09 | 35.71 | 83.14% | / |
| Tryptophan | 0.204% | 73.21 | 43.29 | 36.09 | 82.56% | slightly soluble in water |

The results show that the added amount, calculated based on the molecular weight of the amino acids, does not differ significantly. Among them, glycine and taurine exhibited a redder hue, histidine exhibited a yellowish hue, and methionine had an off odor. The addition of amino acids improved the extraction yield and enhanced the color tone, but the obtained pigment did not achieve a sufficiently red hue.

The above are only the preferred embodiments of the present invention. It should be noted that, for a person skilled in the art, various modifications and refinements can be made without departing from the principles of the present invention, and such modifications and refinements should also be considered within the scope of the present invention.

1. A method for preparing a red Monascus pigment, characterized by comprising the following steps:
step 1: obtaining a Monascus purpureus strain, cultivating, fermenting, acid-precipitating, and performing a first filtration to obtain a precipitate;
step 2: washing the precipitate obtained in step 1, performing a second filtration to obtain a filter cake;
step 3: extracting the filter cake obtained in Step 2 with an ethanol solution, performing peptide powder extraction, and then performing a third filtration to obtain a filtrate;
step 4: performing a fourth filtration of the filtrate obtained in step 3, concentrating, and drying to obtain the red Monascus pigment as a powder.

2. The method according to claim 1, wherein in step 1, a cultivation temperature is between 30° C. and 35° C., and a cultivation time is between 6 and 7 days.

3. The method according to claim 1, wherein in step 1, a fermentation medium comprises: 8 wt % to 10 wt % enzyme-hydrolyzed rice flour, 1 wt % to 3 wt % soybean powder, 0.3 wt % to 1 wt % corn slurry, 0.1 wt % to 0.2 wt % sodium nitrate, 0.1 wt % to 0.3 wt % KH₂PO₄, and 0.05 wt % to 0.3 wt % MgSO₄, and wherein a fermentation temperature is between 30° C. and 35° C., and a fermentation time is between 5 and 8 days.

4. The method according to claim 3, wherein a method for preparing the enzyme-hydrolyzed rice flour comprises mixing rice flour and amylase at a mass ratio of 1:(0.005 to 0.01), performing enzymolysis, cooling, and then adjusting a pH to 3.8 to 4.0 using lactic acid; and
wherein the amylase includes medium-temperature amylase and/or high-temperature amylase, with an enzyme activity of 2000 U/mL, the enzymolysis temperature is between 50° C. and 60° C., and a enzymolysis time is between 30 and 60 minutes.

5. The method according to claim 1, wherein the acid precipitation in step 1 includes adjusting a pH of the fermentation broth to 3 to 3.5 using hydrochloric acid, and an acid precipitation time is 2 to 5 hours; in step 2, a washing process includes mixing the precipitate with an ethanol solution having a volume fraction of 20% to 40% and stirring for 2 to 3 hours.

6. The method according to claim 1, wherein in step 3, during the ethanol solution extraction, a volume fraction of ethanol in the solution is between 70% and 85%, and a mass-to-volume ratio of the filter cake to the ethanol solution is 1:(8 to 10), with an extraction time being between 2 and 3 hours; a peptide powder extraction includes adding peptide powder to the ethanol solution either during or after the ethanol solution extraction, followed by mixing, adjusting a pH to 7 to 9 with sodium hydroxide, and stirring for 5 to 8 hours; and the peptide powder includes at least one of corn oligopeptides, wheat oligopeptides, bovine bone peptides, collagen peptides, hydrolyzed fish peptides, and soybean peptides, and the amount of peptide powder added is 20% to 50% of the weight of the filter cake.

7. The method according to claim 1, wherein in step 4, the fourth filtration is performed using diatomaceous earth filtration, and a concentration process includes reverse osmosis concentration and/or vacuum concentration.

8. A red Monascus pigment obtained by the method according to claim 1, wherein the extraction yield of the red Monascus pigment is >85%; the Lab color values satisfy: L*≥67, a*≥46, b*≥25; after light exposure, the pigment retention rate is >46%; and after high-temperature treatment (120° C., 40 minutes), the retention rate is ≥52%.

9. A red Monascus pigment obtained by the method according to claim 1.

10. A red Monascus pigment obtained by the preparation method according to claim 1, wherein the pigment is applicable as an additive in the preparation of food and/or cosmetics.
